(19) United States
(12) Patent Application Publication
(10) Pub. No.: US 2008/0114396 A1
(43) Pub. Date: May 15, 2008

(54) ADJUSTABLE NON-INVASIVE WOUND CLOSURE SYSTEM

(75) Inventors: John M. Cory, Mystic, CT (US); Keith Levine, Orange, CT (US)

Correspondence Address: LAW OFFICE OF DELIO & PETERSON, LLC., 121 WHITNEY AVENUE, 3RD FLLOR, NEW HAVEN, CT, 06510, US

(73) Assignee: PRECISION CLOSURE LLC, Orange, CT (US)

(21) Appl. No.: 11/559,942

(22) Filed: Nov. 15, 2006

(51) Int. Cl. A61B 17/08

(52) U.S. Cl. 606/216

(57) ABSTRACT

A system for effecting closure of a surgical incision in a patient's body after surgery. The system includes a plurality of closure components to be applied to the patient's skin on each side of the wound and adhered to the skin. A closure connector having an adjustable length secures the closure component tabs of different closure components at a desired spaced, adjustable distance sufficient to close the wound. The closure connector may be a metal clip that is plastically deformable to adjust its length, or may be a pair of plastic connectors which may be adjustably secured to each other by a surgical staple. The connectors may also be hinged to the closure components.

### BACKGROUND OF THE INVENTION

1. Field of the Invention

This invention relates generally to surgical methods and apparatus and more particularly, to apparatus for effecting closure of surgical incisions without using sutures, staples or other invasive devices.

2. Description of Related Art

In closing incisions and other wounds, it is important to both avoid infection and properly align the edges of the skin. Improper re-approximation of the skin at the edges of a surgical incision can interfere with healing and may lead to skin irregularities, excessive scarring and keloiding, i.e., formation of fibrous tumors arising from connective tissue of the skin. Proper alignment becomes more difficult as the length of the incision increases.

U.S. Pat. No. 5,562,705 describes a system of closure units for effecting final closure of a surgical incision in a patients body, each of which includes an elongated alignment template frictionally holding two sets of adhesive coated closure components in aligned opposing relationship and spaced a predetermined first distance from one another along the length dimension of the template for positioning the closure components on opposite sides of the incision for adherence to the patient's skin, which template is removed prior to making the incision, leaving the closure components adhesively secured to the skin in aligned opposing relationship and spaced a first distance from one another transversely of the incision line. The incision is closed by an elongated cover sheet adapted to span the incision line, the cover sheet having therein two sets of paired slots in aligned opposing relationship arrayed each to receive a respective closure component of an opposed pair and spaced from one another along the length dimension of the cover sheet a second distance sufficiently shorter than the first distance as to bring said opposed closure components closer together and draw opposed edges of the incision into alignment and everting contact under slight, but precise, tension. This “tenting” of the joined edges promotes blood flow through connecting tissue of the opposed edges of the incision, resulting in accelerated healing and a thinner scar line.

U.S. patent application Ser. No. 11/529,028 describes a system for effecting closure of a surgical incision in a patient's body after surgery. A plurality of U-shaped closure components having upward extending tabs at the ends and barbs on the underside of the base are held by a tape strip and template applied to the skin so that the closure components are on opposite sides of an incision line. The template is removed, leaving the tape strips and closure components. After surgery is completed, the incision is closed by securing a closure connector comprising a cover sheet or link to a tab on a closure component on one side of the incision, using the closure connector to draw the connected closure component toward a closure component on the other side of the incision, and securing the closure connector to a tab on the closure component on the other side of the incision.

### SUMMARY OF THE INVENTION

While the '705 patent and the '028 application provide accurate closure of a surgical incision, they do not account for adjustment of the degree to which the separated portions of skin on each side of the incision are drawn toward each other and held by the connector. There is a need for such adjustment in order to more precisely align the cut edges of the skin in order to promote better healing. Additionally, there is also a need for adjustment of the edges of the skin during closure of traumatic wounds, where pre-alignment of the skin on either side of the wound is not possible.

Bearing in mind the problems and deficiencies of the prior art, it is therefore an object of the present invention to provide an improved sutureless system for effecting rapid and accurate closure of a surgical incision.

It is another object of the present invention to provide an improved system and method for adjusting alignment of opposed edges of an incision together in a more precise everted relationship, without use of sutures, clamps or other invasive devices.

A further object of the invention is to provide an improved system and method of rapidly aligning and closing an incision and adjusting opposing closure components if necessary.

Still other objects and advantages of the invention will in part be obvious and will in part be apparent from the specification.

The above and other objects, which will be apparent to those skilled in the art, are achieved in the present invention which is directed to a method for effecting closure of a wound in a patient's body comprising providing a plurality of closure components, each having an upward-extending tab, and providing one or more closure connectors having an adjustable length for securing the closure component tabs of different closure components at a desired spaced, adjustable distance. The method includes applying at least one of the closure components over the patient's skin on each side of the wound and adhering the closure components to the skin and closing the wound by securing a closure connector to a tab on a closure component on each side of the wound, and adjusting the length of the closure connector to draw the patient's skin on each side of the wound together to a desired distance sufficient to close the wound. The closure connectors preferably comprise plastically deformable links having ends engageable with the closure component tabs. The closure connectors may comprise a clip or link having a pair of opposed side edges and opposite end portions for engaging the closure component tabs, with the side edges being plastically deformable inward to shorten the length of the closure connector. Such closure connectors may comprise a metal clip or link having a pair of opposed side edges and opposite end portions for engaging a plurality of closure component tabs, or having a pair of opposed side edges and opposite end portions for engaging each closure component tab separately.

Alternatively, the closure connectors may comprise a clip or link having a pair of opposed end portions for engaging the closure component tabs, side edges connected to the end portions, and transverse members connected to the side edges, the transverse members being plastically deformable toward each other to shorten the length of the closure connector.

In another embodiment, the closure connectors each have an upwardly extending tab, which closure connector tabs are adapted to be secured together at a desired spaced distance by a surgical staple.

Preferably, each adjustable closure connector is adapted to secure at least two closure component tabs on each side of the incision.

The method may include using a crimping tool to adjust the length of the closure connector. The crimping tool preferably shortens the length of the closure connector after securing the closure connector to the closure components. The crimping tool may comprises pliers having pressure elements on jaws thereof wherein the pressure elements contacting the closure connector to plastically deform at least a portion of the closure connector and shorten the length thereof.

The closure components are preferably held to the skin by adhesive tape strips, each tape strip having an adhesive on the underside for adhering the tape strip and associated closure components to the skin. The closure components are spaced along each tape strip, and the method includes applying a tape strip and associated closure components on each side of the incision line on the patient's skin to adhere the closure components to the skin.

After securing the closure connector to the closure components, the method may include bending down the tab on at least one of the closure components. The closure components may be configured to secure the closure connector in an elevated position above the wound.

In one embodiment, a closure connector is secured and hinged to at least one of the closure components on one side of the wound prior to adhering the closure components to the skin. The method includes closing the wound by rotating the hinged closure connector to secure the closure connector to a tab on a closure component on the other side of the wound.

Preferably, the closure components are carried on a template prior to application on the patient's skin on each side of the wound. The template includes perforations alongside the closure component tabs, and the method includes removing a portion of the template between the closure component tabs by tearing along the perforations prior to closing the wound.

Where the wound is a surgical incision, the adjustable closure components may be applied to the patient's skin prior to surgery and the closure effected after surgery, or the adjustable closure components may be applied to the patient's skin and the closure effected after surgery. Wherein the wound is a traumatic wound, the adjustable closure components may be applied to the patient's skin and the closure effected after the patient received the wound.

In another aspect, the present invention is directed to a system for effecting closure of a surgical incision in a patient's body after surgery comprising a plurality of closure components, with each having an upward-extending tab. Each closure component is adapted to be applied to the patient's skin on each side of the wound and adhered to the skin. The system also comprises at least one closure connector having an adjustable length for securing the closure component tabs of different closure components at a desired spaced, adjustable distance. The closure connector is adapted to be secured to a tab on a closure component on each side of the wound, and have its length adjusted to draw the patient's skin on each side of the wound together to a desired distance sufficient to close the wound.

The closure connector may comprise a clip or link having a pair of opposed side edges and opposite end portions for engaging the closure component tabs, the side edges being plastically deformable inwardly to shorten the length of the closure connector. Alternatively, the closure connector may comprise a clip or link having a pair of opposed end portions for engaging the closure component tabs, side edges connected to the end portions, and transverse members connected to the side edges, with the transverse members being plastically deformable toward each other to shorten the length of the closure connector. In another alternative, the closure connector may have an upwardly extending tab, which closure connector tab is adapted to be secured together with another closure component tab at a desired spaced distance by a surgical staple.

In a further aspect, the present invention is directed to a method for effecting closure of a wound in a patient's body comprising providing a plurality of closure components, and providing one or more closure connectors for securing the closure components at a desired spaced distance. The one or more closure connector are secured and hinged to at least one of the closure components. The method includes applying at least one of the closure components over the patient's skin on each side of the wound and adhering the closure components to the skin. A closure component on one side of the wound includes a hinged closure connector and a closure component on the other side of the wound is free of a hinged closure connector. The method then includes closing the wound by rotating the hinged closure connector to secure the closure connector to the closure component on the other side of the wound.

The closure connectors are preferably adjustable in length for securing the closure components at a desired, spaced, adjustable distance, and the method includes adjusting the length of the closure connectors to effecting closure of the wound.

In yet another aspect, the present invention is directed to a system for effecting closure of a wound in a patient's body comprising a plurality of closure components, with each closure component adapted to be applied to the patient's skin on each side of the wound and adhered to the skin. At least one closure component for one side of the wound includes a hinge and at least one closure component for the other side of the wound is free of a hinge. The system also includes one or more closure connectors for securing closure components on different sides of the wound at a desired spaced distance. The one or more closure connector are secured and hinged to at least one of the hinged closure components. The hinged closure connector is rotatable to secure the closure connector to at least one closure component free of a hinge on the other side of the wound. Preferably, the closure connectors are adjustable in length for securing the closure components at a desired, spaced, adjustable distance.

Another aspect of the present invention provides a method for effecting closure of a wound in a patient's body comprising providing a plurality of closure components and providing one or more closure connectors for securing the closure components at a desired spaced distance, the closure connectors being adapted to be secured to each other at a desired spaced distance by a surgical staple. The method includes applying at least one of the closure components over the patient's skin on each side of the wound and adhering the closure components to the skin, securing the closure connectors to the closure components on each side of the wound, and closing the wound by applying a surgical staple between closure components on each side of the wound and securing the closure components at a desired, spaced distance.

The closure connectors preferably each have an upwardly extending tab, with the closure connector tabs being adapted to be secured to each other at a desired spaced distance by a surgical staple. The method preferably includes securing the closure connectors to the closure components on each side of the wound with the closure connector tabs proximate each other; and closing the wound by applying a surgical staple between proximate closure connector tabs on opposite sides of the wound.

In a related aspect, the present invention provides a system for effecting closure of a wound in a patient's body comprising a plurality of closure components, with each closure component adapted to be applied to the patient's skin on each side of the wound and adhered to the skin, and one or more closure connectors for securing the closure components at a desired spaced distance. The closure connectors each have an upwardly extending tab, the closure connector tabs being adapted to be secured together at a desired spaced distance by a surgical staple.

### BRIEF DESCRIPTION OF THE DRAWINGS

The features of the invention believed to be novel and the elements characteristic of the invention are set forth with particularity in the appended claims. The figures are for illustration purposes only and are not drawn to scale. The invention itself, however, both as to organization and method of operation, may best be understood by reference to the detailed description which follows taken in conjunction with the accompanying drawings in which:

FIG. 1 is a perspective view of a closure unit constructed in accordance with a first embodiment of the invention.

FIG. 2 is a plan view of the closure unit shown in FIG. 1.

FIG. 3 is a side view of the closure unit of FIG. 2.

FIG. 4 is a top view of a closure component.

FIG. 5 is a side view of the closure component of FIG. 4.

FIG. 6 is a right-end view of the closure component of FIG. 4.

FIG. 7 is a perspective view showing a multiplicity of closure units in place on a patient's body prior to making an incision.

FIG. 8 is a perspective view illustrating a retracted incision or wound, and placement of the closure units relative to its edges.

FIG. 9 is a plan view of one embodiment of a metal clip closure connector to be used to close the incision or wound of FIG. 8.

FIG. 10 is a plan view of another embodiment of a metal clip closure connector to be used to close the incision or wound of FIG. 8.

FIG. 11 is a perspective view of the metal clips of FIG. 9 on the closure components prior to closing the incision of FIG. 8.

FIG. 12 is a perspective view of the metal clips of FIG. 9 after adjustment on the closure components closing the incision or wound of FIG. 8.

FIG. 13 is a side view of FIG. 12 illustrating “tenting” of opposing edges of the skin of a bridged incision or wound.

FIG. 14 is a plan view of one embodiment of the crimping pliers used to adjust the length of the closure connectors of FIG. 9 or 10.

FIG. 15 is a plan view of the pliers of FIG. 14 adjusting the length of the closure connector of FIG. 9.

FIG. 16 is a plan view of another embodiment of the crimping pliers used to adjust the length of the closure connectors of FIG. 9 or 10.

FIG. 17 is a plan view of the pliers of FIG. 16 adjusting the length of the closure connector of FIG. 10.

FIG. 18 is a perspective view of a closure unit constructed in accordance with another embodiment of the invention, employing hinged closure connectors.

FIG. 19 is a perspective view of a closure component having bent back tabs.

FIG. 20 is a side elevational view of a hinged closure connector having elevated closure component tabs to space the closure connector above the closed wound incision.

FIG. 21 is a plan view of a further embodiment of a metal clip closure connector to be used to close an incision or wound, in its undeformed state.

FIG. 22 is a plan view of the metal clip closure connector of FIG. 21 in its crimped, plastically deformed state.

FIG. 23 is a side elevational view of another embodiment of the closure unit of the present invention, wherein a surgical stapler is used to adjustably space the closure connectors and components to close the wound incision.

FIG. 24 is a plan view of the stapled closure connectors and closure components of FIG. 23.

### DESCRIPTION OF THE PREFERRED EMBODIMENT(S)

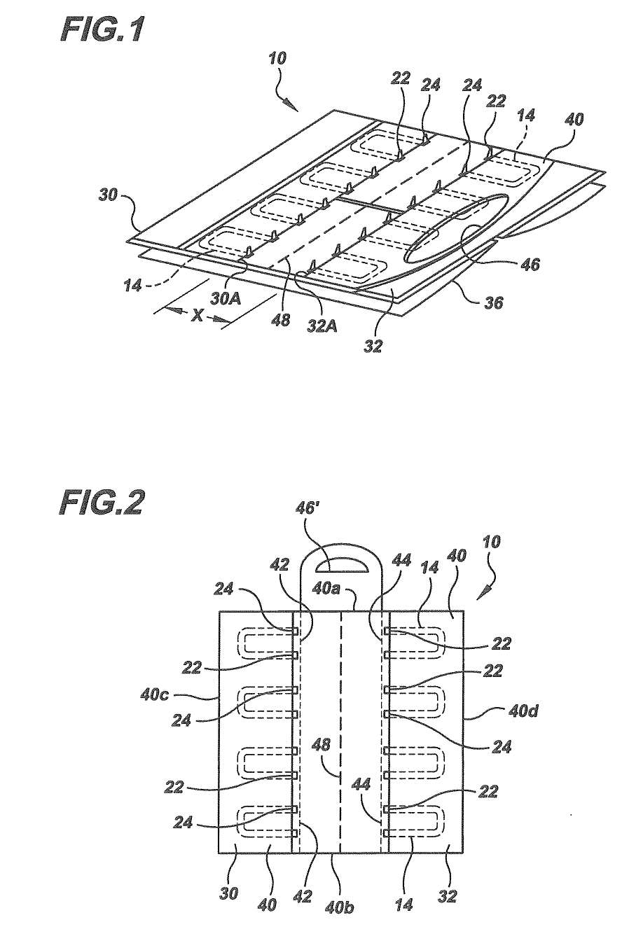

In describing the preferred embodiment of the present invention, reference will be made herein to FIGS. 1-24 of the drawings in which like numerals refer to like features of the invention.

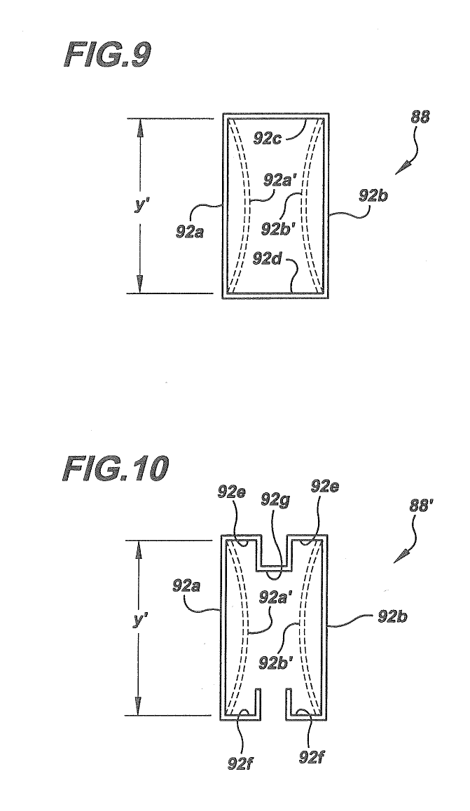

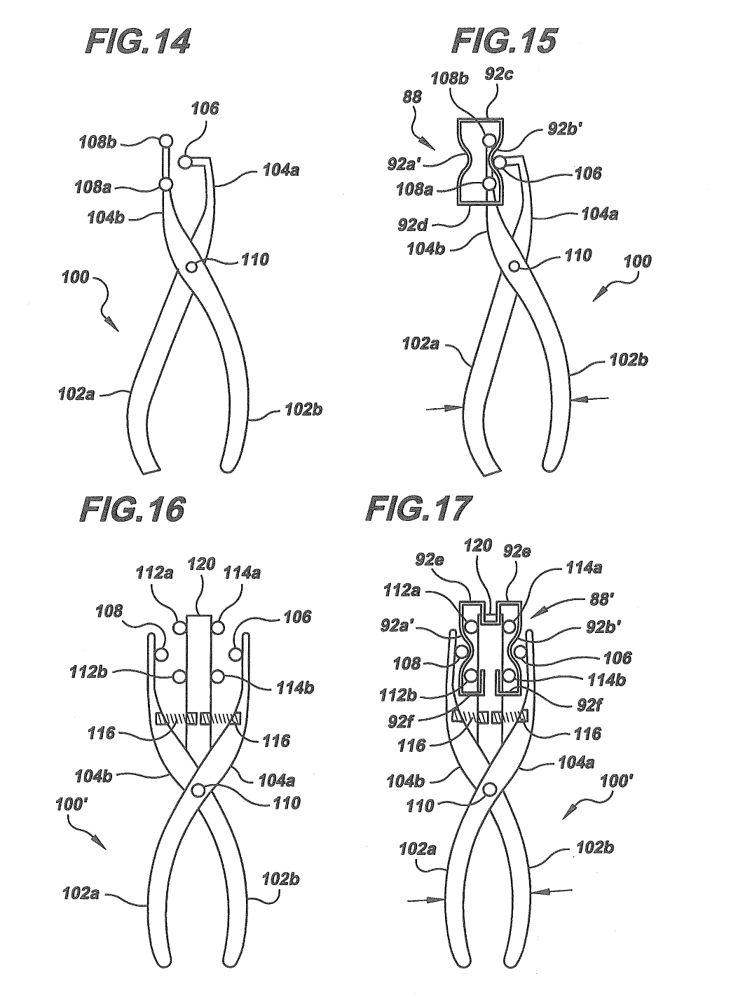

Referring to the drawings, the wound closure system in accordance with the invention consists of a closure unit 10 shown in FIGS. 1-3, 7, 8 and 11-13, each of which preferably includes a plurality of closure components 14 on opposite sides of an incision or wound. Closure unit 10, is similar to that shown in U.S. patent application Ser. No. 11/529,028, the disclosure of which is hereby incorporated by reference. Adjustable closure components, such as the metal links or clips 88 and 88′ shown in FIGS. 9 and 10, coact with opposed closure components 14 and have their lengths adjusted, preferably by the crimping pliers of FIGS. 14-17, to bring the edges of the skin on either side of an incision or wound into the desired everting contact. The closure components and closure connectors are preferably separately packaged in sterile, peelable envelopes, (not shown) similar to that used to package plastic bandage strips.

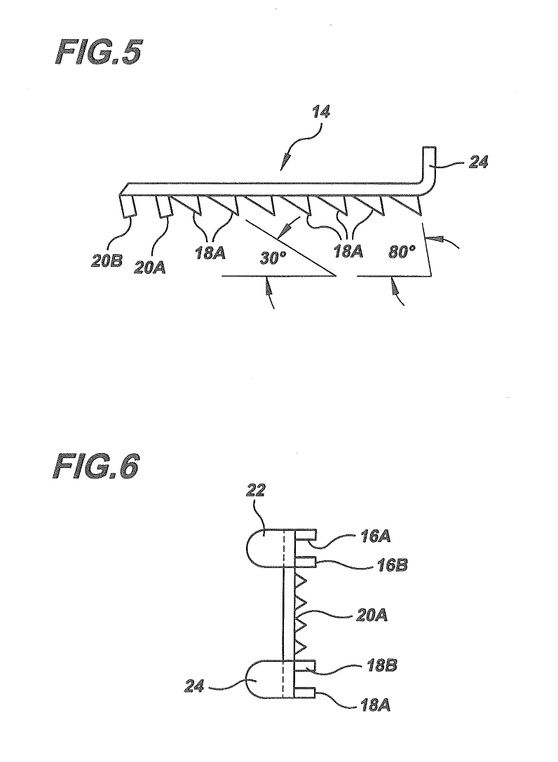

Referring to the drawings each closure component, one of which is shown at 14 in FIGS. 4-6, is in one embodiment generally “U”-shaped in plan view having spaced parallel arms 16 and 18 joined at one end by a cross-member 20 and have integral upstanding tabs 22 and 24 at their respective extremities. Optionally, for greater strength, a second cross-member 20′ may be added between arms 16, 18 near the open ends of the U. The underside surface of each of arms 16 and 18 and of cross-member 20 is preferably smooth, and the tape (described further below) is used to ensure that the closure components 14 are secured to the patient's body. Optionally, the underside of each closure component may be provided with two spaced parallel rows of barbs or stickers 16A, 16B, 18A 18B, 20A, 20B, respectively, which as seen in FIG. 5, are inclined toward the tabs 22 and 24. The leading and trailing edges of each barb are preferably inclined at angles of 80 degrees and 30 degrees, respectively, relative to the upper surface of the arms. The closure components may be stamped from thin sheet aluminum or stainless steel typically 0.020 in. (0.50 mm) thick, and typically are 0.50 in. (13 mm) long and 0.281 in. (7.2 mm) wide. The arms and cross-member are each 0.062 in. (1.6 mm) wide, arms 16 and 18 are spaced 0.156 in. (4 mm) apart, and tabs 22 and 24 extend upwardly 0.062 in. (1.6 mm) from the top surface of their respective arms. The tabs are rounded at their upper ends to facilitate placement thereover of openings in the cover sheet during closure of an incision, and are sufficiently malleable to be bent over, if desired, after closure.

Referring to FIGS. 1-3, a typical closure unit 10 includes eight “U”-shaped closure components 14 retained in two parallel rows of four equally spaced components each by respective strips of adhesive tape 30 and 32 which overlie the upper surfaces of a respective row of closure components. The inner edges 30A and 32A of the two tapes abut a respective row of upstanding tabs, the tape being sufficiently wide, typically 0.75 in. (19 mm), to completely cover and extend beyond the cross-members 20 of the aligned closure components so as to insure their adhesion to the patient's skin. One tape that may be used for this application is an elastic nonwoven tape available in either tan or white available from 3M Company, Cat. No. 9907, which has bidirectional elasticity so as to be exceptionally conformable. It is hypoallergenic, breathable and hydrophobic, making it useful for attaching devices to the skin. This proprietary tape is made up of polyurethane elastic nonwoven backing coated with hypoallergenic super sticky pressure sensitive acrylate adhesive. The tapes may alternatively be made of Mylar or polyethylene of about 0.005 in (0.13 mm) thickness. During manufacture, after the tape strips are applied over the precisely aligned closure components, the adhesive coated surfaces are covered with a silicone-coated paper release liner 36.

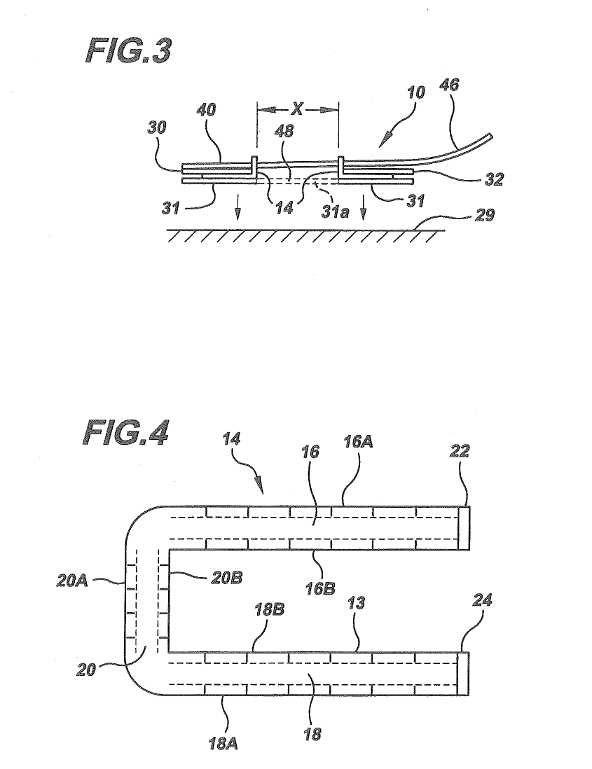

In one embodiment of the invention, adhesive coating on the underside of tapes 30, 32 secure the rows of closure components 14 to the patient's skin, so that the undersides of the closure components directly contact the skin. In another embodiment shown in FIG. 3, a lower tape layer 31 sandwiches closure component arms between it and the upper tape layers 30, 32, so that the closure components are adhered to, but do not directly contact, patient's skin 29. The lower tape layer has an adhesive layer on its underside and optionally contains a center section 31a, between the tabs of the opposed rows of closure components 14, that is preferably clear and without adhesive to permit initial placement and perforated to permit easy removal prior to surgery. Center section 31a may include a target line 48 equidistant from the closure component tabs to align the closure unit 10 with the desired skin incision line.

In accordance with one aspect of the invention opposing upstanding tabs 22, 24 in the two rows of closure components 14 established by tapes 30 and 32 are held in alignment, and at a predetermined spacing between the rows, by a discardable plastic template 40, punched to provide two opposed rows of eight slits each, for receiving and holding the upstanding tabs 22, 24 of a respective four-component group of closure components. The slits are uniformly distributed across the width of template 40, typically about 2 in. (50 mm), and the outermost slits in each row are spaced from respective edges 40a and 40b of the template by approximately one-half the spacing between components so that when two or more closure units are affixed side-by-side to a patient's skin, all will be evenly distributed along the length of the incision. The center portion of template 40 preferably has perforations 42, 44 (FIG. 2) along and inside the line of tabs 22, 24, and a transverse line 48, preferably dotted, is printed thereon equidistant from and parallel to the two rows of perforations 42, 44. The center portion of template 40 between perforation rows 42 and 44 may be torn off, as discussed further below. Preferably, at least this center template portion is transparent. Template ends 40c and 40d are preferably substantially perpendicular to template edges 40a and 40b and may be coincident with the far edges of tapes 30, 32, as shown in FIG. 2, or may be within those tape edges, as shown in FIG. 1. Optional finger holes 46 (FIG. 1) and/or 46′ (FIG. 2) are provided at an end or side of the template for facilitating handling and removal. Finger hole 46′ is preferred to remove the center template 40 between perforation rows 42 and 44.

The two rows of slits for receiving the upstanding tabs of the opposed closure components are precisely spaced apart by a distance “X”, and preferably equidistant from line 48. Typically, the dimension X may be 19 mm which, when line 48 is placed over the line on the patient's skin at which the incision is to be made, locates the tabs 22, 24 9.5 mm to either side of the incision line.

Template 40 may be fabricated from thin transparent sheet plastic having a thickness of 0.005 in. (0.13 mm), preferably Mylar, a polyethylene terephalate marketed by E.I. duPont de Nemours, which has been biaxially oriented and heat set to provide dimensional stability and adequate strength.

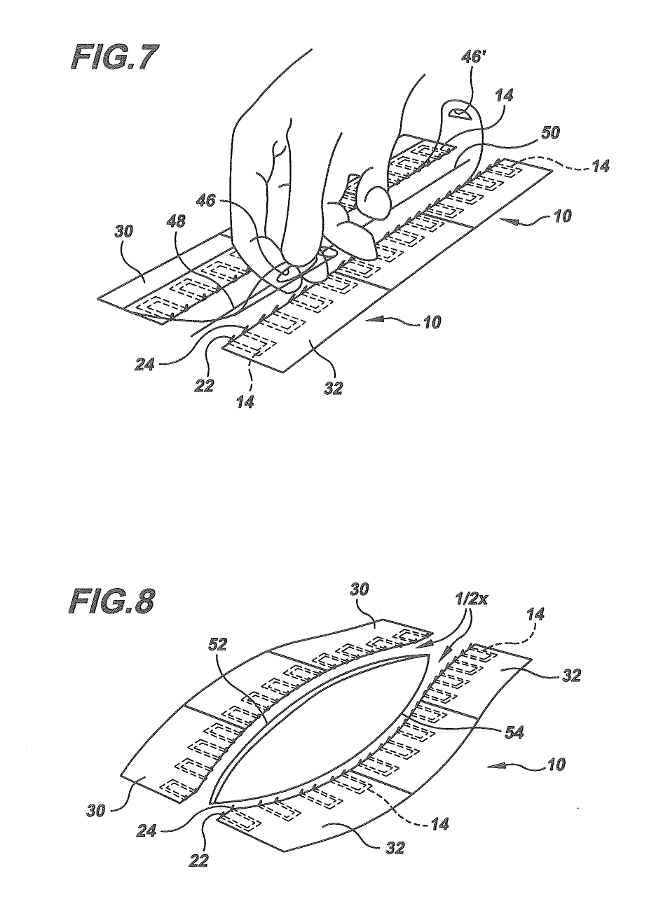

When the closure system of the present invention is to be used in connection with planned surgery, prior to making the incision the area of a patient's body surrounding the planned incision is conventionally prepped by shaving and sterilization. The surgeon marks the epidermis along the intended length of the incision, typically with a blue line 50, and then selects that number of packages of closure units 10 which, when placed side-by-side along the line, will exceed the length of the planned incision. For example, if the planned length is five inches, the surgeon may call for at least three and possibly four closure units to take care of the possibility that a longer incision may be needed As pictorially represented in FIG. 7, after peeling the silicone-coated paper release liner 36 from tapes 30 and 32 to expose the adhesive coating of the tapes, and the undersides of the eight closure components of a closure unit 10, the surgeon positions the template 40 with transverse dotted line 48 in alignment with the intended incision line 50, thereby precisely placing the opposed tabs 22, 24 of the two rows of closure components 14 substantially equidistantly from the incision line and, more importantly at the distance X from one another.

After the template is properly positioned, the surgeon presses the tapes 30 and 32, including the areas disposed between the aims 16 and 18 of the side-by-side closure components 14, into contact with the patient's skin. If barbs are employed on the undersides of the closure components, they do not penetrate, but only depress the skin sufficiently to strongly resist movement of the closure components in the direction of inclination of the barbs. By this simple procedure, the surgeon easily, rapidly and accurately affixes the opposed closure components to the patient's skin in precise alignment on opposite sides of the incision line and at a fixed and constant distance X from one another.

This procedure is repeated until the required number of closure units has been applied to the patient's skin in side-by-side proximity, three in the example depicted in FIG. 7, following which the templates 40 are removed in turn. In the illustration, the templates have already been removed from two of the units, and the surgeon's hand is shown removing the template from the third unit in its entirety by grasping the finger hole 46 and lifting it to first release the row of tabs held in place by tape 32, followed by release of the other row of tabs. More preferably, only the center section of the template between the closure component tabs 22, 24 is removed by grasping finger hole 46′ and lifting upward, thereby tearing the template center section off along perforated rows 42, 44 (FIG. 2). Left behind are two rows of closure components firmly adhered to the skin along opposite sides of the intended incision, each row in the example shown in FIG. 7 having twelve uniformly spaced components, and twenty-four tabs 22, 24 uniformly spaced about 0.25 in. (6.4 mm) apart along the 6 in. (15 cm) overall length of the three units.

The surgeon then makes the incision along the line 50 through the skin, sub-cutaneous tissues and muscle as necessary to expose the surgical site, after which the opposed tissue layers may, if necessary, be retracted as shown in FIG. 8. Alternatively, the incision may be made through the transparent templates 40, along the dotted line 48, and then at least the central portion of the templates are removed by tearing along perforation rows. Because the uniformly spaced closure components are firmly adhered to the skin with bidirectionally elastic tape, the skin can be retracted or stretched up to fifty percent of the length of the incision without fear of displacement of the closure components. The tapes 30 and 32 stretch as necessary to conform to the stretching of the skin. Since the components are spaced from edges 52 and 54 of the incision, they are not affected by, nor do they interfere with, the use of refractors or other instruments or procedures attendant the surgery. After the surgical procedure is completed in conventional fashion by suturing the fascial and sub-cuticular layers, leaving only the dermis for closing, the edges of the incision are reapproximated and, if called for, blotted dry with sterile gauze.

The wound closure system of the present invention may be also be used to close traumatic wounds or incisions without having to pre-locate the closure connectors prior to the wound or incision having occurred. In such case, a desired number of the closure units are applied opposite one another on either side of the wound opening, with the distance of the upstanding tabs 22, 24 of each of the closure unit tapes 30, 32 to the respective edges 52, 54 of the wound opening being approximately one-half (½) “X” (FIG. 8).

At the desired time during surgery, the epidermis is closed with a multiplicity of closure connectors, such as links or clips 88 (FIG. 9) or 88′ (FIG. 10), which are preferably made of metal or such other material that is plastically deformable. Clips 88, 88′ are more preferably made of stainless steel wire of approximately 0.020 in. (0.5 mm) thickness. Clip 88 is rectangular in shape and has continuous end members 92c, 92d extending between opposite ends of side members 92a, 92b. The width of clip 88 is such that each end of clip 88 can be secured over two adjacent closure component tabs 22, 24. Clip 88′ is “H” shaped and has two segmented end members 92e and 92f on each end between side members 92a, 92b. Intermediate member 92g connects end segments 92e, while end segments 92f are unconnected and have an end opening therebetween. The width of clip 88′ and the width of each end segment 92e, 92f is such that the end segments may be secured over adjacent closure component tabs 22, 24.

Each clip has a length “Y′” between the inside of end members (92c, 92d for clip 88 and 92e, 92f for clip 88′) that may be greater or less than distance X when the side members 92a, 92b are straight. The distance Y′ is preferably equal to X+D₁, where D₁ is any additional clearance needed to go over the closure component tabs 22, 24. The length of the clips, in this case the distance between the ends, is adjustable, for example by bending the side members 92a, 92b by lateral pressure directed inwards until they plastically deform to positions 92a′, 92b′, respectively. Because of the bending or crimping of the sides, the length of each clip then becomes less than the initial length Y′. The clips may be made in different lengths for different surgical applications. Multiple pairs of clips can be pre-packaged for speed and convenience, for example, four pair of clips in a popular size to cover most incision lengths. Alternatively, clip length Y′ may be set to be used without crimping, at a length X-A, where A is the distance reduction required to bring the edges of the wound into everting or “tenting” contact, as discussed further below in connection with FIG. 13.

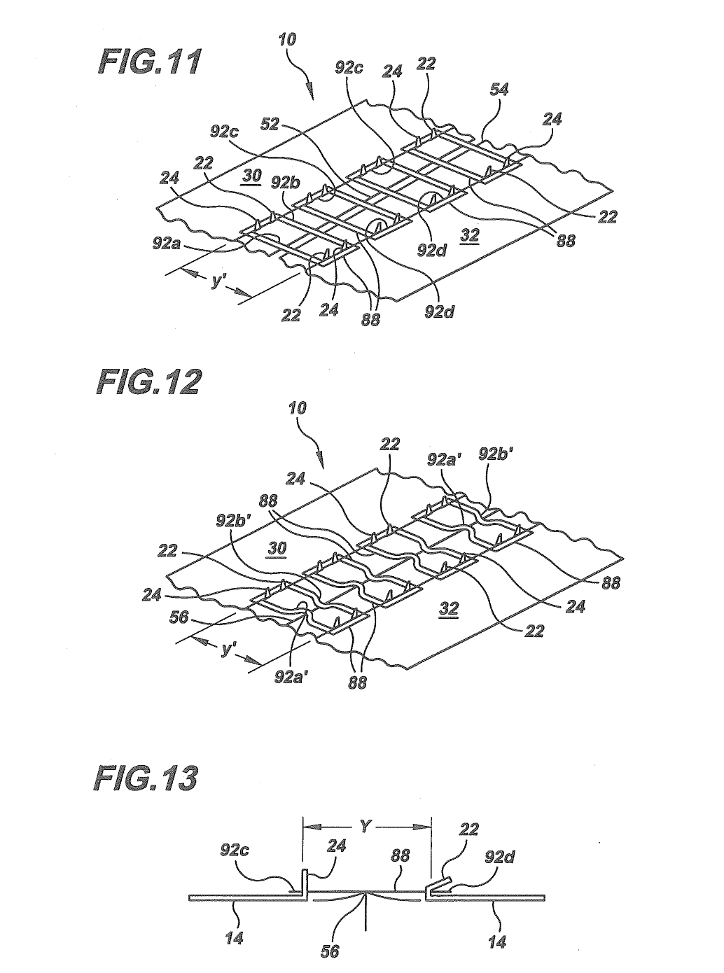

To effect closure of the edges 52, 54 of the incision or wound, undeformed clips 88 or 88′, shown in FIGS. 9 and 10, respectively, are preferably used to draw the adhered closure component tabs on either side of the wound opening toward each other. For example, starting at one end of the incision or wound, the surgeon grasps clip 88 so that the upstanding tabs 22 and 24 of a closure component on one side of the wound are received within one end 92c of the clip. The surgeon then grasps the opposite end 92d of the clip and pulls it toward the closure component on the other side of the wound. As shown in FIG. 11, once one edge 52 of the skin incision is drawn to the vicinity of the other edge 54, clip end 92b is dropped down to engage tabs 22 and 24 in the opposite closure component. The surgeon may use his hand to hold end 92b of clip 88, or may use any other suitable grasping means such as a pair of needle-nose pliers or other tool. When clip 88′ is employed, the ends 92e, 92f may be used in a manner analogous to that described for ends 92c, 92d to bring the skin edges in closer vicinity to each other. Additional closure connectors are used in the manner described above until a sufficient number of closure components have been bridged to cover the length of the incision or wound. The surgeon's opposite hand may be used to provide additional assistance in closing the incision, by urging the opposite sides of the skin together.

After securing the closure connectors over both opposite sets of tabs, the distance between edges 52 and 54 of the incision or wound is determined. If there still exists a space between skin edges 52 and 54, as shown in FIG. 11, the length of the closure connectors is adjusted. In the case of the clips 88 or 88′, the opposite sides are urged against each other until the sides 92a, 92b become plastically deformed and the skin edges 52, 54 are in sufficient contact 56, as shown in FIG. 12. Because tapes 30 and 32 preclude movement of opposed closure components 14 toward one another, adjustment of the length of the closure connectors achieves with precision the desired everting contact of the edges of the incision by reason of the span Y between the tabs of opposing closure components. For cases where the closure components were applied as a unit prior to surgery, distance Y is typically about 2 mm less than the distance X initially established between them prior to making the incision. Because the length of each closure component is individually adjustable, different clips may be adjusted to different lengths and the opposite skin edges may be closed to the desired everting contact along the entire length of the closed incision or wound.

The structural and physical properties of the closure connectors are selected so that, after adjustment of the length, they are essentially inelastic, and thus unstretchable, within the range of forces present trying to open the close wound. The adjustable closure connectors bring respective opposed closure components closer together, by a predetermined precise amount, than they were when initially adhered to the skin, sufficiently to bring the edges of the wound into everting or “tenting” contact 56, as shown in FIG. 13, under a slight, but precise amount of tension. Closure of an incision several inches long can be done in a few minutes, considerably less time than is required for suturing and comparable to stapling, but with a better eventual result.

Before or after adjusting the lengths of the closure connectors, the surgeon bends the tabs in a direction away from the incision, as shown by the arrows in FIG. 13, for retaining the closure connector clips 88 in place. Preferably, opposing tabs on both sides of the incision are bent in this manner after the cover sheet is deposed over the closure components. The arcuate shape of the upper ends of tabs 22 and 24 allows the opposite ends of the closure connectors to readily slip over and engage the tabs, permitting rapid and accurate closing of the wound.

Preferred tools to bend the clip sides 92a, 92b and adjust the lengths of the clips are shown in FIGS. 14-17. In FIGS. 14 and 15, plier-like crimping tool 100 has handles 102a, 102b on opposite ends of jaw members 104a, 104b, respectively, connected at pivot 110. Pressure element 106 is disposed on the end of jaw member 104a, and pressure elements 108a and 108b are spaced apart on jaw member 104b, respectively below and above pressure element 106. When one side of a closure connector clip 88 is placed between the opposed pressure elements and the handles 102a and 102b are squeezed together as shown in FIG. 15, the clip side is plastically deformed, as shown by side 92b′, by the force of element 106 deforming the side between elements 108a and 108b. As described above, the bending of the clip sides causes the length to contract, and the amount of length adjustment may be varied by the pressure and deformation of the clip sides. Using tool 100, each side of the clip has to be individually deformed to produce crimped sides 92a′ and 92b′.

An alternative crimping tool 100′ is depicted in FIGS. 16 and 17 crimping clip 88′. Instead of having to crimp each side of the clip individually, tool 100′ is able to crimp both sides simultaneously. Each jaw has a single pressure element, 106 on jaw 104a and 108 on jaw 104b. An elongated central member 120 has a pair of pressure elements on either side, with elements 112a and 112b respectively above and below jaw pressure element 108 and elements 114a and 114b respectively above and below jaw pressure element 106. Springs 116 disposed between member 120 and jaws 104a and 104b urge the jaws apart. As shown in FIG. 17, by placing clip 88′ between the pressure elements of the central member and jaws, and squeezing the jaw handles 102a, 102b together, the jaw pressure elements 106 and 108 crimp the clip sides 92b′ and 92a′, respectively, inward between the central member pressure elements and shorten the length of the clip.

Either tool 100, 100′ may be used to crimp either clip 88, 88′. As an alternative to the aforementioned crimping tools, any other tools or means may be employed to bend the clip sides and shorten the clip length. While it is preferred that the length of the clips be adjusted after the clips are in place over the closure component tabs as shown in FIGS. 11 and 12, it is possible to at least partially adjust the length of the clips prior to placing them over the opposing tabs of the closure components.

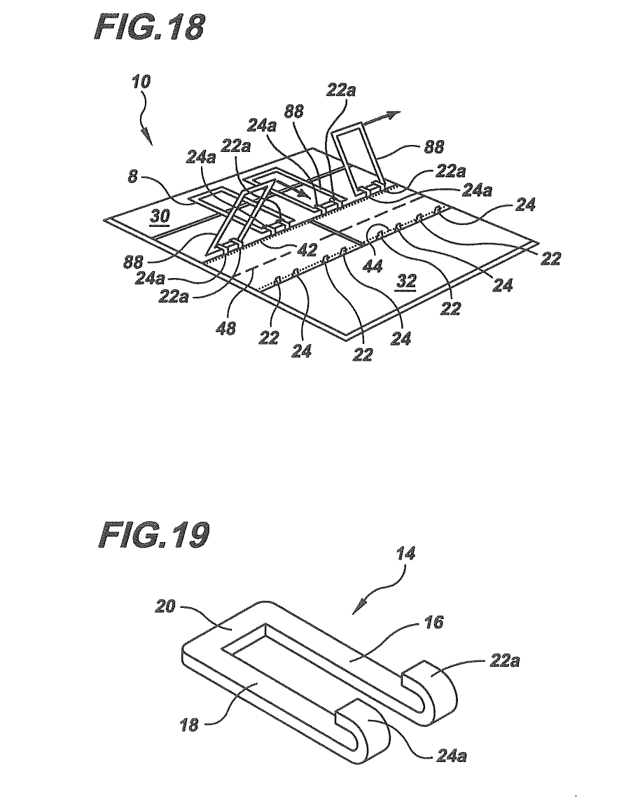

In another embodiment, the closure connectors may be hinged to the closure components on one side of the wound. As shown in FIG. 18, one of the end members of each of clips 88 is pre-secured to tabs 22a, 22b of each of the closure components extending beneath tape 30, The tabs 22a, 24a are bent back toward the opposite end of closure component 14, as shown in FIG. 19, to hold the clip end members and act as a hinge. Initially, the clips are provided over tape already rotatably secured to tabs 22a, 22b, but disposed flat on the tape, prior to being secured to the patient's skin. When the incision is ready to be closed, the clips are rotated upward and over toward the opposite closure connector tabs 22, 24, in the direction of the arrow (FIG. 18), and secured to the opposite tabs. The clips may then be crimped in the manner described above to adjust the lengths of the closure connectors and the contact of the opposing skin edges. Tabs 22a, 22b in FIG. 19 may also be used with separate, non-hinged closure connectors of the type shown in FIGS. 11 and 12 to secure the wound.

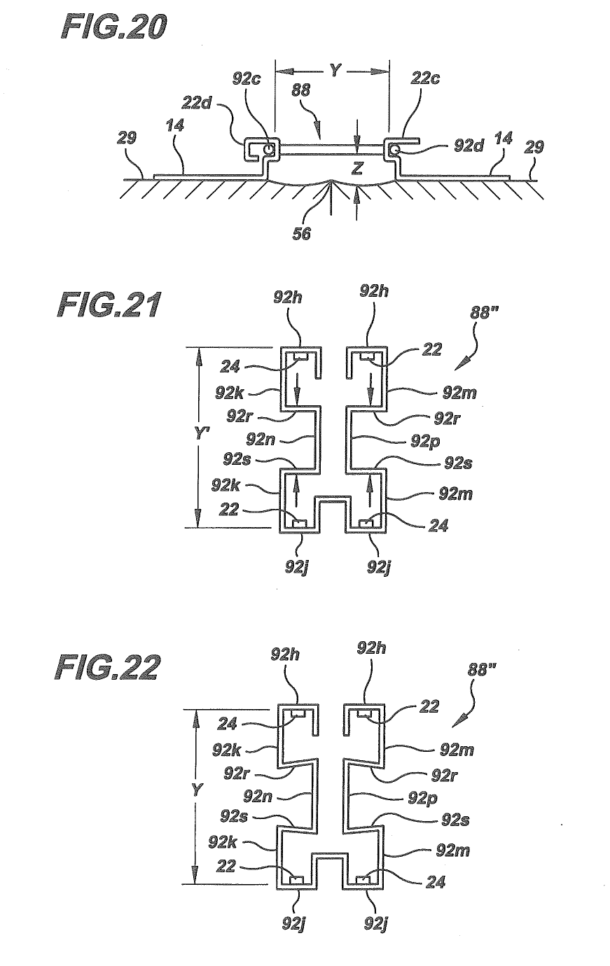

An alternate hinged closure connector is shown in FIG. 20, wherein bent tabs 22c, 22d are configured to elevate clip 88 above the skin 29 by distance “z.” Tab 22d is bent to form a substantially closed hinge around clip end member 92c, and opposite tab 22c is bent to permit insertion and securing of clip end member 92d when the wound is to be closed. After securing, the clip may then be crimped in the manner described above to adjust the lengths to bring the opposing skin edges into everting contact 56. The elevation of the closure connector promotes better healing of the closed incision by avoiding contact with clip 88. Instead of being hinged, clip 88 (or 88) may be secured in the elevated position shown by use of tabs 22c on both sides of the wound.

FIGS. 21 and 22 depict another embodiment of the closure connector clip or link, here shown as clip 88″, Undeformed clip 88″: in FIG. 21 has length Y′ between the insides of opposite end members 92h and end members 92j. Segmented end members 92h are secured over closure component tabs 22, 24 on one side of the wound, and segmented end members 92j are secured over closure component tabs 22, 24 on the other side of the wound. On the sides, opposite end members 92h, 92j are connected to segmented side members 92k, 92m, respectively. On the left side, segmented side members 92k are connected by inward extending members 92r and 92s, which are themselves connected by center member 92n. On the right side, segmented side members 92m are connected by inward extending members 92r and 92s, which are connected by another center member 92n. The use of members 92r, 92s transverse to the segmented side members connected to the end members permits a squeezing force, applied as shown by the arrows to the transverse members, to bring segmented sides 92k, 92m and segmented end members 92h, 92j closer together, and reduce the initial undeformed distance Y′ between the end members 92h, 92j. Conventional needle nose pliers may be used to apply the squeezing forces via the jaw members, first on one side and then on the other side, to bring spaced transverse members 92r, 92s toward each other to crimp and plastically deform clip 88″.

The configuration after plastic deformation of clip 88″ is shown in FIG. 22. The angles between segments 92k, 92r, 92n, 92s and 92k on one side, and 92m, 92r, 92p, 92s and 92m on the other side are bent from their initial right angles to acute angles, resulting in the shortening of the clip between end members 92h and 92j to distance Y to draw the opposite closure components tabs 22, 24 closer together to provide the desired degree of contact between opposite skin edges. As with clips 88 and 88′, clip 88″ may also be pre-secured and hinged to tabs 22a, 22b in the manner described in connection with FIGS. 18, 19 and 20, or used with separate, non-hinged closure connectors.

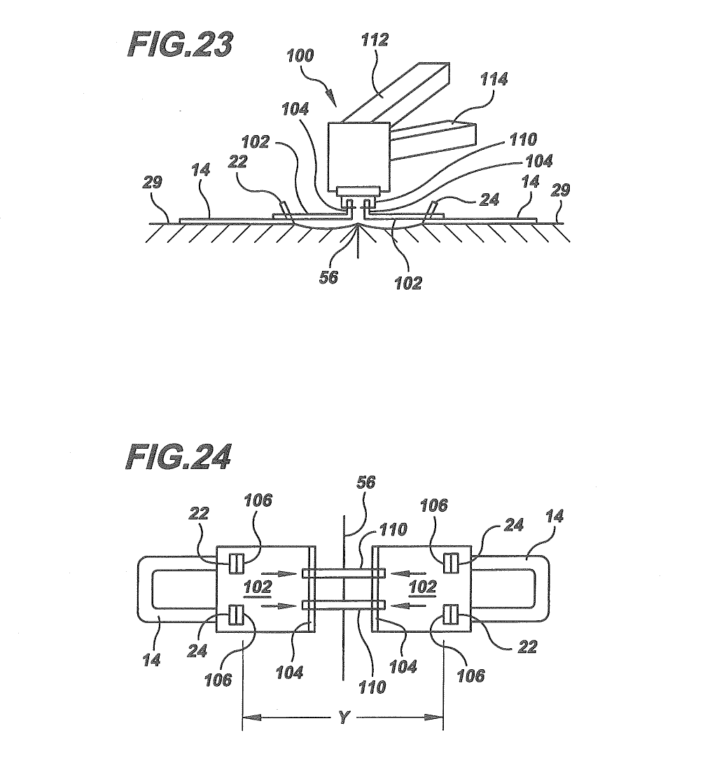

Another system and method for effecting closure of a wound in a patient's body is shown in FIGS. 23 and 24, where closure connectors in the closure unit depicted are able to use existing adjustable surgical staplers to pull the incision or wound together. Closure connectors 102 each have a flat base with openings 106 at one end to receive tabs 22, 24 from closure components 14, and an upstanding tab 104 at the opposite end. When the component connector openings 106 are secured over closure component tabs 22, 24 on opposite sides of the wound, with the tabs 104 proximate each other, tabs 104 may be drawn toward each other and secured at a desired distance apart by otherwise conventional surgical staples 110. The closure connectors 102 are preferably made from a polymer such as polytetrafluoroethylene (PTFE) which is stiff and strong enough to retain its shape, but soft enough to be penetrated by a staple. A surgical stapler 100 (FIG. 23), such as that taught in U.S. Pat. No. 4,204,623, the disclosure of which is hereby incorporated by reference, may be used to apply staples 110 so that the staple legs laterally penetrate the tab 104 walls. As the stapler handles 112, 114 are squeezed together, the legs of staple 110 are crimped by stapler 100 toward each other and move the closure connector tabs 104 closer together in the directions shown by the arrows FIG. 24) to adjust the spacing of the tabs 104. The final spacing of tabs 104 results in distance Y (FIG. 24), which is selected between tab openings 106 to achieve a desired degree of contact 56 between the wound edges. The closure connectors 102 may also be hingedly secured to bent tabs of closure components 14 (either flat or elevated), as shown in FIGS. 18-20, on either side of the wound. The staples employed in the closure unit embodiment of FIGS. 23 and 24 connect only the closure connectors, and never actually enter, or even contact, the patient's skin.

Seven to ten days after the operation, by which time the incision will have healed more completely than would be the case if sutures or staples were used which often are removed within four or five days following surgery, the connectors bridging the incision are removed, e.g., cover sheets 60, links 88, 88′ 88″, or connectors 102, and the tapes are swabbed with a solution which dissolves their adhesive coating and allows the tapes and the adhered closure components to be peeled from the surgical area. If employed, each barb of the closure component may produce a small red depression in the patient's skin, this is believed to disappear within a few minutes after it is removed.

While specific values of the dimensions X and Y have been indicated, they are intended to be exemplary only and may be varied within reasonable limits so long as the dimension Y is sufficiently less than dimension X that the closure connector cover sheet or link brings opposed edges of the incision into everting contact under slight tension. Normally, the X dimension would remain at 19 mm and the closure connector clips or links produced with a variety of Y′ dimensions to provide a range of differences to accommodate the system to variations in the tenting properties of the skin of different individuals.

Additionally, the closure connectors may be configured to engage any number of closure component tabs on each side of the incision. While the previous description shows ends capable of securing two adjacent tabs on the closure component, each closure connector may connect one tab on each side of the incision, or more than two such tabs on each side of the incision.

Thus, the present invention provides an improved sutureless system for effecting rapid and accurate closure of a surgical incision to enabling subsequent facile and rapid adjustment of opposed edges of an incision together in an everted relationship, without use of sutures, clamps or other invasive devices.

While the present invention has been particularly described, in conjunction with a specific preferred embodiment, it is evident that many alternatives, modifications and variations will be apparent to those skilled in the art in light of the foregoing description. It is therefore contemplated that the appended claims will embrace any such alternatives, modifications and variations as falling within the true scope and spirit of the present invention.

Thus, having described the invention, what is claimed is:

1. A method for effecting closure of a wound in a patient's body comprising:
providing a plurality of closure components, each having an upward-extending tab;
providing one or more closure connectors having an adjustable length for securing the closure component tabs of different closure components at a desired spaced, adjustable distance;
applying at least one of the closure components over the patient's skin on each side of the wound and adhering the closure components to the skin;
closing the wound by securing a closure connector to a tab on a closure component on each side of the wound, and adjusting the length of the closure connector to draw the patient's skin on each side of the wound together to a desired distance sufficient to close the wound.

2. The method of claim 1 wherein the closure connectors comprise plastically deformable links having ends engageable with the closure component tabs.

3. The method of claim 1 wherein the closure connectors comprise a clip or link having a pair of opposed side edges and opposite end portions for engaging the closure component tabs, the side edges being plastically deformable inward to shorten the length of the closure connector.

4. The method of claim 3 wherein the closure connectors comprise a metal clip or link having a pair of opposed side edges and opposite end portions for engaging a plurality of closure component tabs.

5. The method of claim 3 wherein the closure connectors comprise a metal clip or link having a pair of opposed side edges and opposite end portions for engaging each closure component tab separately.

6. The method of claim 1 wherein the closure connectors comprise a clip or link having a pair of opposed end portions for engaging the closure component tabs, side edges connected to the end portions, and transverse members connected to the side edges, the transverse members being plastically deformable toward each other to shorten the length of the closure connector.

7. The method of claim 1 wherein the closure connectors each have an upwardly extending tab, which closure connector tabs are adapted to be secured together at a desired spaced distance by a surgical staple.

8. The method of claim 1 wherein each adjustable closure connector is adapted to secure at least two closure component tabs on each side of the incision.

9. The method of claim 1 including using a crimping tool to adjust the length of the closure connector.

10. The method of claim 1 including using a crimping toot to shorten the length of the closure connector, after securing the closure connector to the closure components.

11. The method of claim 10 wherein the crimping tool comprises pliers having pressure elements on jaws thereof, the pressure elements contacting the closure connector to plastically deform at least a portion of the closure connector and shorten the length thereof.

12. The method of claim 1 wherein the closure components are held to the skin by adhesive tape strips, each tape strip having an adhesive on the underside for adhering the tape strip and associated closure components to the skin, the closure components being spaced along each tape strip, and including applying a tape strip and associated closure components on each side of the incision line on the patient's skin to adhere the closure components to the skin.

13. The method of claim 1 including bending down the tab on at least one of the closure components after securing the closure connector thereto.

14. The method of claim 1 wherein the closure components secure the closure connector in an elevated position above the wound.

15. The method of claim 1 wherein a closure connector is secured and hinged to the at least one of the closure components on one side of the wound prior to adhering the closure components to the skin, and including closing the wound by rotating the hinged closure connector to secure the closure connector to a tab on a closure component on the other side of the wound.

16. The method of claim 1 wherein the closure components are carried on a template prior to application on the patient's skin on each side of the wound, the template including perforations alongside the closure component tabs, and including removing a portion of the template between the closure component tabs by tearing along the perforations prior to closing the wound.

17. The method of claim 1 wherein the wound is a surgical incision, and the adjustable closure components are applied to the patient's skin prior to surgery and the closure is effected after surgery.

18. The method of claim 1 wherein the wound is a surgical incision, and the adjustable closure components are applied to the patient's skin and the closure is effected after surgery.

19. The method of claim 1 wherein the wound is a traumatic wound, and the adjustable closure components are applied to the patient's skin and the closure is effected after the patient received the wound.

20. A system for effecting closure of a surgical incision in a patient's body after surgery comprising:
a plurality of closure components, each having an upward-extending tab, each closure component being adapted to be applied to the patient's skin on each side of the wound and adhered to the skin; and
at least one closure connector having an adjustable length for securing the closure component tabs of different closure components at a desired spaced, adjustable distance, the closure connector being adapted to be secured to a tab on a closure component on each side of the wound, and have its length adjusted to draw the patient's skin on each side of the wound together to a desired distance sufficient to close the wound.

21. The system of claim 20 wherein the closure connector comprises a clip or link having a pair of opposed side edges and opposite end portions for engaging the closure component tabs, the side edges being plastically deformable inwardly to shorten the length of the closure connector.

22. The system of claim 20 wherein the closure connector comprises a clip or link having a pair of opposed end portions for engaging the closure component tabs, side edges connected to the end portions, and transverse members connected to the side edges, the transverse members being plastically deformable toward each other to shorten the length of the closure connector.

23. The system of claim 20 wherein the closure connector has an upwardly extending tab, which closure connector tab is adapted to be secured together with another closure component tab at a desired spaced distance by a surgical staple.

24. A method for effecting closure of a wound in a patient's body comprising:
providing a plurality of closure components;
providing one or more closure connectors for securing the closure components at a desired spaced distance, the one or more closure connector being secured and hinged to at least one of the closure components;
applying at least one of the closure components over the patient's skin on each side of the wound and adhering the closure components to the skin, a closure component on one side of the wound including a hinged closure connector and a closure component on the other side of the wound being free of a hinged closure connector; and
closing the wound by rotating the hinged closure connector to secure the closure connector to the closure component on the other side of the wound.

25. The method of claim 24 wherein the closure connectors are adjustable in length for securing the closure components at a desired, spaced, adjustable distance, and including adjusting the length of the closure connectors to effecting closure of the wound.

26. A system for effecting closure of a wound in a patient's body comprising:
a plurality of closure components, each closure component adapted to be applied to the patients skin on each side of the wound and adhered to the skin, at least one closure component for one side of the wound including a hinge and at least one closure component for the other side of the wound being free of a hinge; and
one or more closure connectors for securing closure components on different sides of the wound at a desired spaced distance, the one or more closure connector being secured and hinged to at least one of the hinged closure components, the hinged closure connector being rotatable for secure the closure connector to the at least one closure component free of a hinge on the other side of the wound.

27. The system of claim 26 wherein the closure connectors are adjustable in length for securing the closure components at a desired, spaced, adjustable distance.

28. A method for effecting closure of a wound in a patient's body comprising:
providing a plurality of closure components;
providing one or more closure connectors for securing the closure components at a desired spaced distance, the closure connectors being adapted to be secured to each other at a desired spaced distance by a surgical staple;
applying at least one of the closure components over the patient's skin on each side of the wound and adhering the closure components to the skin;
securing the closure connectors to the closure components on each side of the wound; and
closing the wound by applying a surgical staple between closure components on each side of the wound and securing the closure components at a desired, spaced distance.

29. The method of claim 28 wherein the closure connectors each having an upwardly extending tab, the closure connector tabs being adapted to be secured to each other at a desired spaced distance by a surgical staple; and including securing the closure connectors to the closure components on each side of the wound with the closure connector tabs proximate each other; and closing the wound by applying a surgical staple between proximate closure connector tabs on opposite sides of the wound.

30. A system for effecting closure of a wound in a patient's body comprising:
a plurality of closure components, each closure component adapted to be applied to the patient's skin on each side of the wound and adhered to the skin; and
one or more closure connectors for securing the closure components at a desired spaced distance, the closure connectors each having an upwardly extending tab, the closure connector tabs being adapted to be secured together at a desired spaced distance by a surgical staple.
